(19) United States
(12) Patent Application Publication
(10) Pub. No.: US 2009/0227762 A1
(43) Pub. Date: Sep. 10, 2009

(54) NOVEL CATALYTIC MATERIALS AND THEIR USE IN THE PREPARATION OF POLYMERIC MATERIALS

(75) Inventors: Richard Simon Blackburn, North Yorkshire (GB); Christopher Mark Rayner, West Yorkshire (GB); Christopher Martin Pask, West Yorkshire (GB); Patrick Columba McGowan, West Yorkshire (GB)

Correspondence Address: MOORE & VAN ALLEN PLLC, P.O. BOX 13706, Research Triangle Park, NC, 27709, US

(73) Assignee: UNIVERSITY OF LEEDS, Leeds (GB)

(21) Appl. No.: 12/092,010

(22) PCT Filed: Oct. 31, 2006
(86) PCT No.: PCT/GB06/04059

§ 371 Date: Sep. 19, 2008

(30) Foreign Application Priority Data
Oct. 31, 2005 (GB) 0522154.4

(51) Int. Cl. C09B 45/00; C07F 5/06; C07D 213/81; C08G 63/06; C08G 63/08; C07D 277/62

(52) U.S. Cl. 528/354; 534/701; 534/713; 556/176; 546/323; 528/361; 534/707; 548/156

(57) ABSTRACT

The present invention provides a catalyst for use in the preparation of a coloured polymeric material, said catalyst comprising a coloured organometallic compound. Preferably, said catalyst comprises a metal such as aluminium and at least one organic chromophore, such as an azo chromophore, said chromophore being either directly bonded to said metal, or indirectly bonded to said metal through a ligand. The invention also envisages a method for the preparation of a coloured polymer, the method comprising performing a polymerisation reaction in the presence of such a catalyst. The method is particularly applicable to the preparation of poly(lactic acid), and offers significant benefits over the processes of the prior art, both economically and environmentally.

### FIELD OF THE INVENTION

The present invention is concerned with novel catalyst materials which find application in the synthesis of polymeric materials. In a particularly preferred embodiment, the invention provides catalysts which facilitate the preparation of coloured polymeric materials in which the shade and strength of colour can be closely controlled. The invention also provides novel polymeric materials and methods for their preparation.

### BACKGROUND TO THE INVENTION

In view of the rapid worldwide depletion of petrochemical feedstocks, attention has increasingly turned to the production of new, useful and environmentally friendly polymers which would offer a more sustainable future. Interest has focused particularly on materials such as poly(lactic acid) (PLA), which is a linear aliphatic thermoplastic polyester derived from 100% renewable sources, such as corn and sugar beet. Furthermore, the polymer has the advantage of being biodegradable.^{1,2}

Initial uses of this material have, however, been limited to biomedical applications such as sutures³ and drug delivery systems⁴, in view of its limited availability and relatively high cost of manufacture. More recently, large-scale operations for the economic production of PLA polymer used for packaging and fibre applications have been developed by NatureWorks LLC (USA).

The use of such fibres in fabric for apparel applications is an important development for several reasons, one of the most significant of which is the fact that polyesters currently used for apparel applications, mainly poly(ethyleneterephthalate) (PET), account for over 40% of world textile consumption (which is second only to cotton), and their use is constantly increasing. The production of such polyesters consumes fossil fuel resources, and subsequent disposal of the polymer adds to landfill sites, since they are non-biodegradable and are not easily recycled. By way of contrast, PLA fibre is derived from annually renewable crops, it is 100% biodegradable, and its life cycle potentially reduces the carbon dioxide level in the earth's atmosphere.

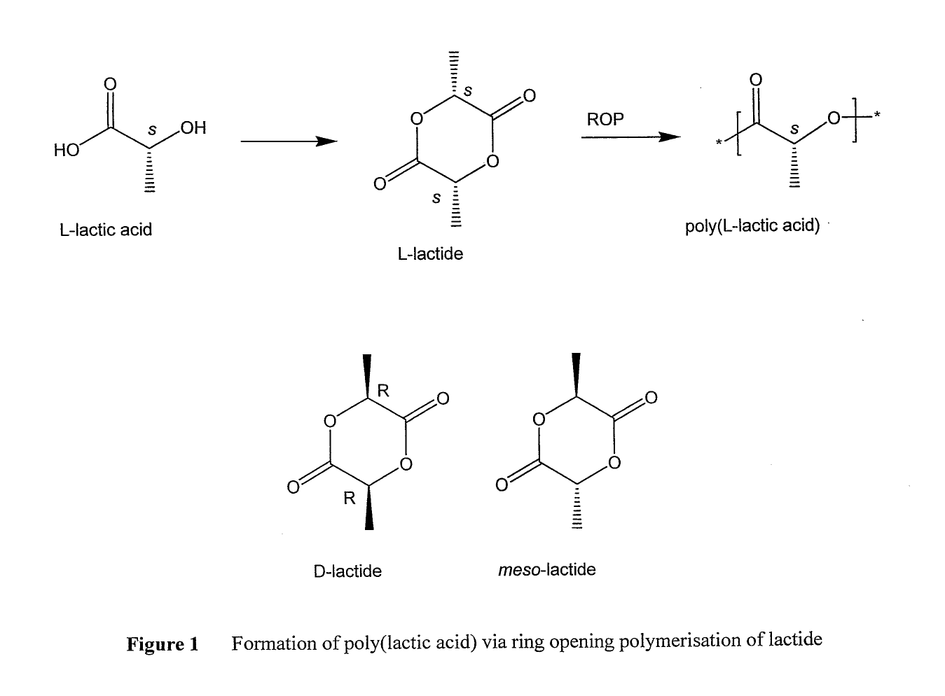

The production of PLA does, in fact, use 20-50% less fossil resources than comparable petroleum-based fibres.⁵ PLA is typically produced by milling a renewable resource, such as corn, and separating starch, from which dextrose is processed and then subsequently converted to lactic acid through fermentation.^{5, 6} The polymer is then formed either by direct condensation of lactic acid, or via the cyclic intermediate dimer (lactide) through a ring opening polymerisation (ROP) process, as illustrated in FIG. 1.¹ The latter process provides the most effective and versatile method for the preparation of PLA.

The lactide precursors can exist as three different stereoisomers (L-lactide, D-lactide and meso-lactide), as shown in FIG. 1. The lactide stereochemistry can have an important impact on the polymerisation process, and the respective PLAs, once formed from the different lactide precursors, can have different physical and mechanical properties, including rates of degradation. For example, isotactic poly(L-lactide) (PLLA) is a semicrystalline polymer with a melting transition near 180° C., whereas atactic poly(rac-lactide) and poly(meso-lactide) are amorphous polymers.⁷ Lactic acid derived from fermentation processes consists of 99.5% L-isomer, and this material has been the subject of earlier studies.⁸

The ring-opening polymerisation (ROP) of lactide has been the subject of investigation for over a century.⁹ The reaction may be promoted by the addition of a variety of catalytic materials, with several metal-containing species finding particular application in this regard. Metal alkoxides are the most commonly used of such species for the ring-opening polymerisation of cyclic esters, and simple sodium, lithium, and potassium alkoxides can be used for this purpose. However, the high basicity of these ionic species can lead to side reactions, such as epimerisation of chiral centres in the polymer backbone.

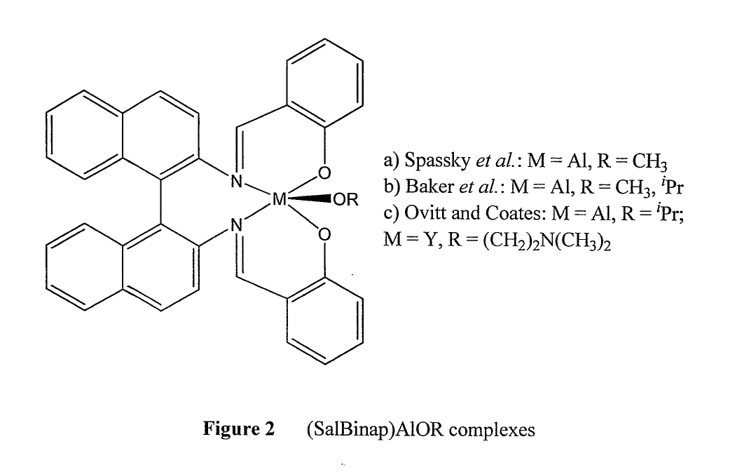

Other metal alkoxides are much more selective in this regard, and therefore find more widespread use. Initiators such as aluminium alkoxides,¹⁰ yttrium and lanthanide alkoxides¹¹ and, more recently, iron alkoxides¹² have been shown to give a controlled and living polymerisation of lactides via a so-called coordination-insertion mechanism. The majority of aluminium complexes that have been reported contain so-called salen/salan ligands. Several aluminium Schiff base catalysts have been successfully exploited for the stereoselective ROP of rac-lactide. In particular, Spassky et al.¹³ discovered that ((R)-SalBinap)-AlOMe (FIG. 2) could polymerise rac-lactide to crystalline PLA with higher melting temperatures (187° C.) than ‘optically pure PLA’. Since then, Baker et al.¹⁴ and Coates^{15, 16} have reported the polymerisation of rac-lactide with rac-(SalBinap)AlOⁱPr.

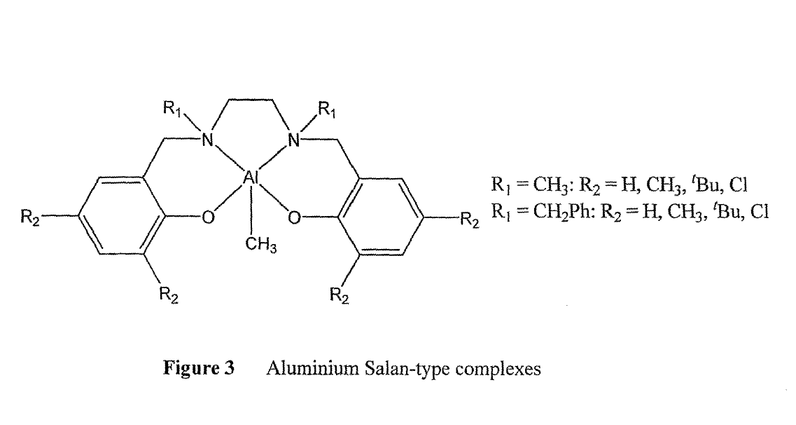

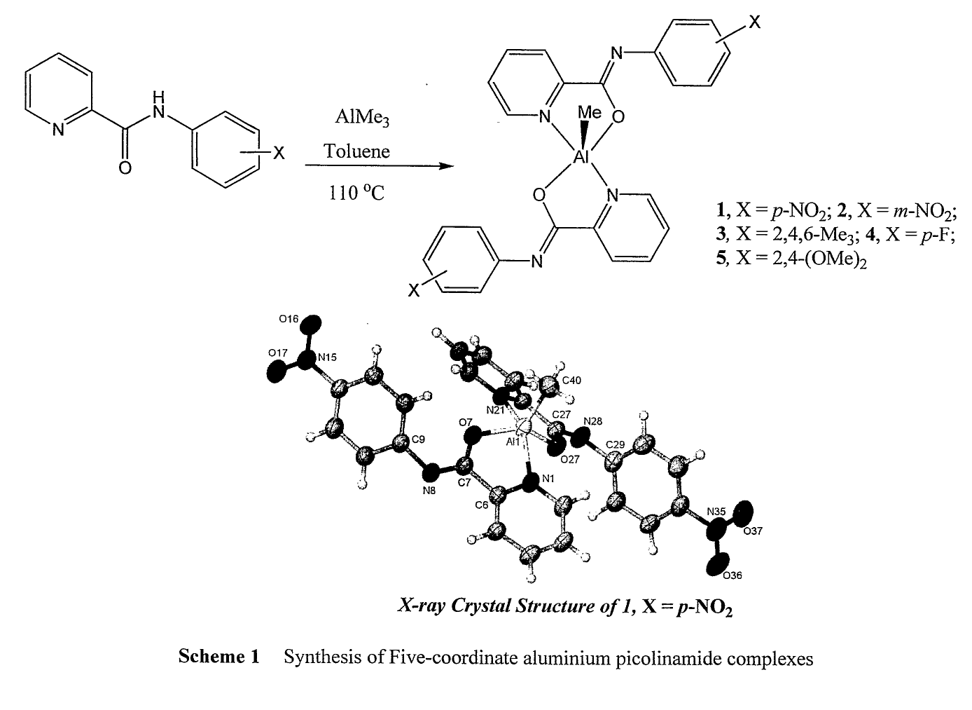

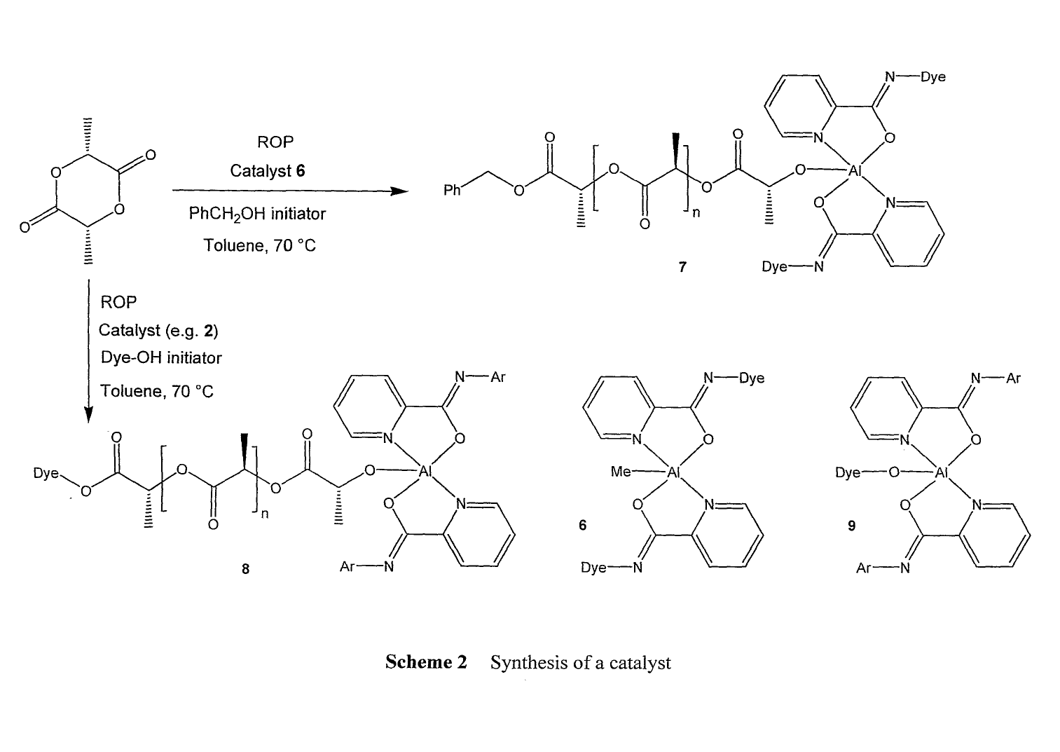

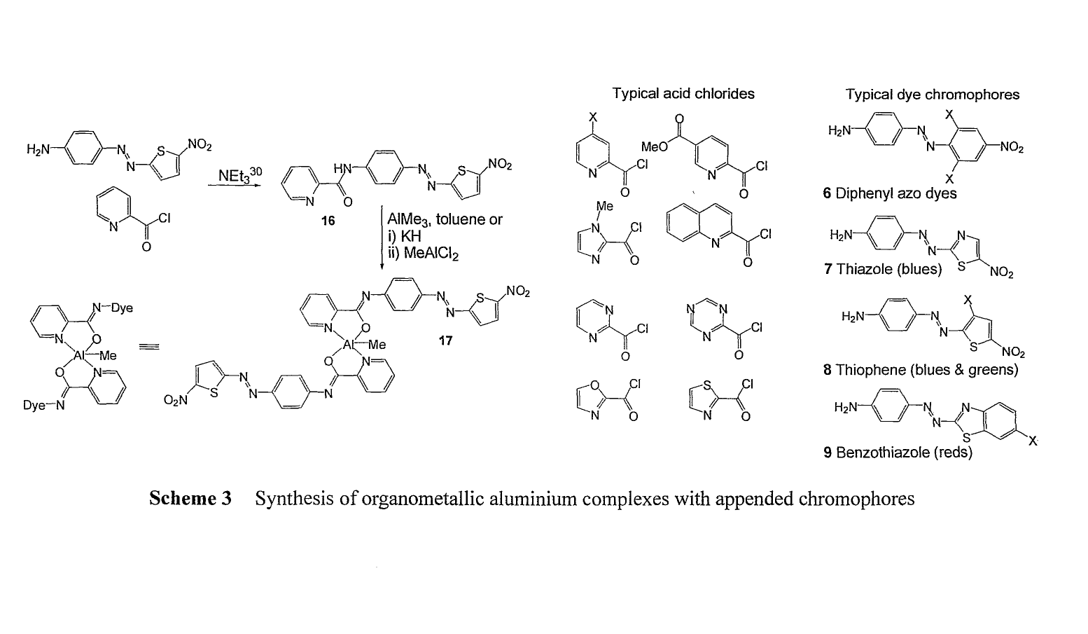

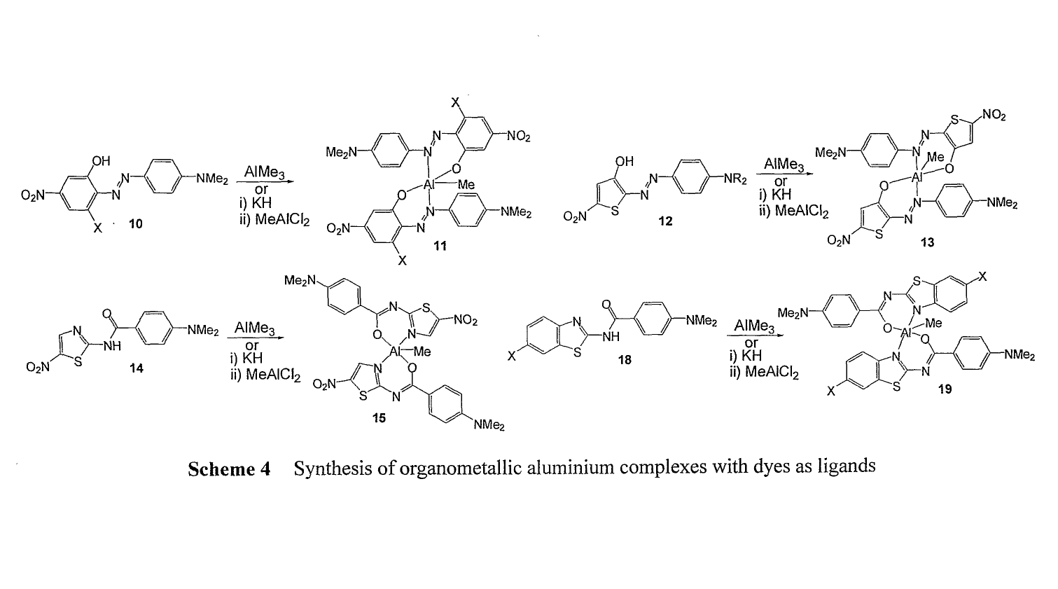

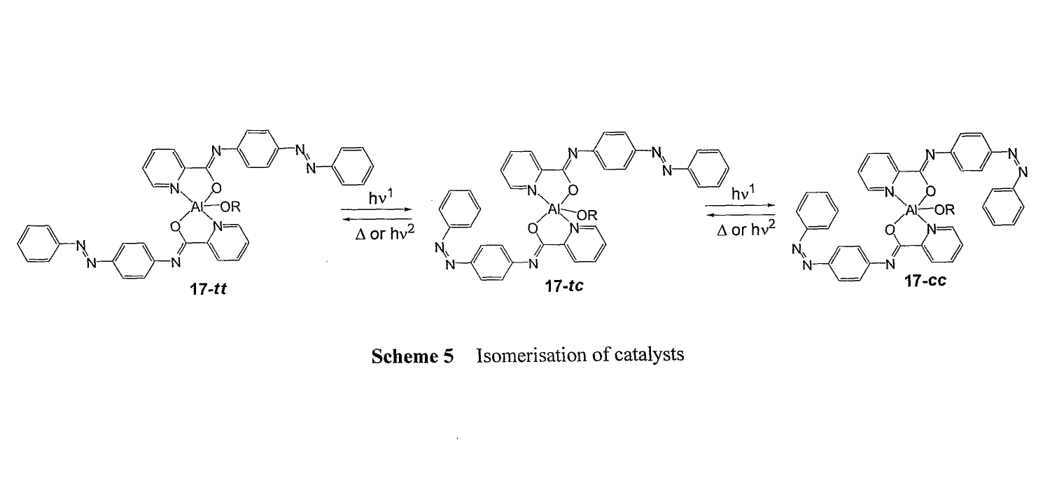

More recently, Gibson has reported a new family of aluminium catalysts (FIG. 3), stabilised by tetradentate phenoxyamine (salan-type) ligands, which have been shown to display an unprecedented degree of stereocontrol in the polymerisation of rac-lactide.¹⁷ The PLA produced ranged from highly isotactic to highly heterotactic, depending on the ligand substituents. Gibson has also reported the [5-Cl-salen]AlOCH₃ complex, which behaves as a room temperature initiator for the controlled polymerisation of D,L- and L-lactides due to the electron withdrawing substituents present on the Schiff base ligand backbone.¹⁸ The majority of the work carried out by Gibson et al involved salen/salan-type ligands, or derivatives thereof, but other recent workers in this field have reported the use of non-salen/salan ligands involving four-, five- and six-coordinate aluminium compounds.¹⁹⁻²²

Thus, several options are available from the prior art for the preparation of PLA. However, as the range of potential applications of this material continues to grow, other difficulties become apparent. Specifically, the proposed use of PLA fibres in fabric for apparel applications has the consequence that coloration of the material becomes a significant issue, since it is required for most large-scale (tonnage) applications.

Various options are, of course, available in this regard, with the dyeing of PLA presenting an obvious approach which is currently being investigated in Leeds^{23, 24} and elsewhere.^{1, 5} Dyeing invariably involves adaptation of methods applicable to the coloration of PET, and significant success has been achieved using this approach. However, there can be drawbacks, since the melting point of the polymer and its acid/alkaline hydrolysis stability can prove to be problematic in typical coloration processes. It has been shown^{1, 5}, for example, that temperatures above 110° C. are required to achieve suitable dyebath exhaustion due to the crystalline nature of the polymer. Ideally 130° C. (the typical temperature for PET coloration) would be used to achieve efficient coloration of PLA but, at this temperature, the fibre undergoes significant strength and elongation loss during wet processing, as is shown by the data in Table 1.²⁵ Additionally, the optimum dyeing pH is around 7, but as the results in Table 2 demonstrate, increasing pH leads to alkaline hydrolysis, which results in strength and elongation loss in PLA. It is clear, therefore, that a coloration process that can achieve high colour strength without exposing the fibre to these potentially damaging conditions is essential for future commercial applications of PLA, and this problem has been one area of interest for the present inventors.

TABLE 1
Effect of dyeing temperature on tensile strength loss and elongation loss
of PLA

| | Tensile strength loss | |
|---|---|---|
| Temp. of dyeing (° C.)^{a} | (%) | Elongation loss (%) |
| 70 | 8.5 | 0.0 |
| 80 | 12.3 | 0.0 |
| 90 | 16.0 | 0.0 |
| 100 | 18.5 | 0.0 |
| 110 | 40.5 | 20.2 |
| 120 | 56.5 | 66.3 |
| 130 | 100.0 | 100.0 |

^{a}Dyed with 2% omf C.I. Disperse Blue 79 for 90 minutes at pH 4

TABLE 2
Effect of pH of dyeing on tensile strength loss and elongation loss of PLA

| pH of dyeing^{a} | Tensile strength loss (%) | Elongation loss (%) |
|---|---|---|
| 4 | 40.5 | 20.2 |
| 5 | 34.6 | 2.6 |
| 6 | 40.0 | 3.9 |
| 7 | 58.0 | 59.6 |
| 8 | 59.4 | 71.5 |

^{a}Dyed with 2% omf C.I. Disperse Blue 79 for 90 minutes at 110° C.

As is well known, the range of molecules used in the dyeing process is wide and varied and, amongst the vast numbers of different materials which are available, the use of metals in the dyeing is common. Thus, in addition to dye structures incorporating co-ordinated metals for colour production, many dye types require pre- or post-treatment with a metal salt. Nearly all natural dyes require application with a mordant (typically using salts of Cr, Sn, Zn or Cu) in order to achieve sufficient wash and light fastness and to provide satisfactory levels of dye exhaustion.

However, for obvious reasons, dyeing with the use of mordants such as Co, Sn or Cr salts leads to problems due to the effluent released from the dyeing process, in view of the waste water limits defined for the concentrations of heavy metals.²⁶ As a consequence, research has been conducted into dyeing with natural dyes using Al, Cu, or Fe(II) sulphate as mordants,²⁷ with particular emphasis being placed on salts of Al and Fe, which are considered to have significantly lower environmental impact than other heavy metal counterparts, and this consideration has been particularly relevant to the work of the present inventors (vide infra).²⁶

### SUMMARY OF THE INVENTION

The present invention has particularly been directed towards the development of a new range of materials which are suitable for catalysing the ring opening polymerisation of lactides, and which allow for considerable modification of the steric and electronic properties of the ligand framework and, hence, polymerisation activity.²² As a consequence, a range of materials has been produced which is suitable for this purpose, but which also finds application in the catalysis of numerous polymerisation reactions, and provides particularly effective results when employed in the production of polyesters. In addition, some materials have been developed which address the need for the efficient coloration of various fibres used in fabric for apparel applications, and which are especially useful in relation to polyester fibres and, most particularly, PLA fibres.

Thus, according to a first aspect of the present invention, there is provided a catalyst for use in the preparation of a radiation absorbing polymeric material, said catalyst comprising a radiation absorbing organometallic compound, wherein the wavelength of maximum absorption of each of said radiation absorbing polymeric material and said radiation absorbing organometallic compound lies in the region of from 200-1200 nm.

Hence, the present invention envisages radiation absorbing polymeric materials and radiation absorbing organometallic compounds which have a wavelength of maximum absorption in the infra-red, visible, and/or ultra-violet regions of the electromagnetic spectrum. Particularly favourable results are obtained in the preparation of coloured polymeric materials using coloured organometallic compounds.

In the context of the present invention, the term coloured is to be interpreted as having a wavelength of maximum absorption which lies within the visible wavelength region of 400-700 nm, and a catalyst according to the first aspect of the invention would comprise an intrinsically coloured compound which fulfilled this criterion.

Typically, the method of preparation of said radiation absorbing polymeric material comprises a polymerisation reaction and said catalyst comprises a polymerisation catalyst.

Organometallic compounds according to the invention comprise at least one organic chromophore, which is the chemical moiety which absorbs radiation, and at least one metal atom. Suitable metals in the context of the invention include aluminium, together with the transition metals and the metals of the lanthanide and actinide series. Particularly favourable results are achieved with aluminium, titanium, zirconium, scandium, hafnium, vanadium and iron, but the most favoured metal is aluminium, partly in view of its ready availability, relatively low cost and non-toxic nature.

Virtually any chromophore is suitable for incorporation in the catalysts according to the present invention provided that the chromophore comprises means for attachment to the metal atom, said means for attachment comprising a suitable binding site. The chromophore absorbs radiation in at least one of the infra-red, visible and ultra-violet regions of the electromagnetic spectrum. Amongst suitable chromophores in this context may be mentioned azo compounds, di- and tri-arylmethane compounds, methine, polymethine and azomethine derivatives, anthraquinone compounds, phthalocyanine derivatives, and various xanthene, acridine, azine, oxazine, thiazine, indamine, indophenol, aminoketone, hydroxyketone, nitro, nitroso, quinoline, stilbene and thiazole compounds, as well as certain carbocyclic and heterocyclic derivatives well known to those skilled in the art. Chromophores which absorb radiation in the visible region of the spectrum are disclosed in the Colour Index published by the Society of Dyers and Colourists, and available online at http://www.colour-index.org. Particularly favourable results are achieved with azo compounds.

Preferably, the organometallic compounds according to the first aspect of the invention comprise metal complex compounds wherein the metal atom is attached to at least one ligand. Most preferably, said organometallic compounds are coloured compounds of the general formula (A):

MLₓD_{y} (A)

wherein
D represents a chromophoric group;
M represents a metal atom;
L represents a non-chromophoric ligand;
x=0-8; and
y=1-9.

The values of x and y are determined by virtue of the identity and oxidation state of the metal, and the relevant co-ordination geometry. The non-chromophoric ligand L does not contribute significantly to the desired radiation absorption, since it does not absorb to any significant extent at the specific wavelength of the required application.

Typically, the metal atom is attached to two ligands. The radiation absorbing chromophore may optionally comprise the at least one ligand which is attached to the metal atom, and thereby be directly bound to the metal atom as, for example, in compounds of formula (B) and (C). Alternatively, the chromophore may be attached to the at least one ligand and, as a consequence, be indirectly bound to the metal atom via the non-chromophoric ligand, such as in compounds of formula (D). In a further embodiment, the catalyst may comprise both direct and indirect linkages, as in the compounds of formula (E).

D-M-D (B)

D-M-L (C)

D-L-M-L-D (D)

D-L-M-D (E)

In these formulae, D, M and L have the meanings ascribed to them above and the multiple D and L groups in compounds (B), (D) and (E) may be the same or different, and may comprise groups D¹, D² and L¹, L², respectively, so the compounds may be more conveniently represented as follows:

D¹-M-D² (B-1)

D¹-L¹-M-L²-D² (D-1)

D¹-L-M-D² (E-1)

wherein D¹ and D² represent chromophoric groups which may be the same or different;
M represents a metal atom; and
L¹ and L² represent non-chromophoric ligands which may be the same or different.

When the chromophore absorbs radiation in the visible region of the spectrum, the compounds of formula (B) are generally found to provide coloured catalysts which provide a darker and duller range of hues.

The ligands are bound to the metal atoms by means of suitable pendant linking groups of the sort which are well known to those skilled in the art, typical examples being nitrogen and oxygen-containing groups, such as amino groups and hydroxy groups. The ligand, when it does not comprise the chromophore per se, but is linked to the chromophore, may comprise any organic residue, but typically comprises an aryl or heteroaryl residue which includes a linking group by means of which the chromophore may be attached. Preferred examples of aryl residues include phenyl, naphthyl, anthracyl and phenanthryl groups, whilst suitable heteroaryl residues include a range of heterocycles which comprise at least one nitrogen and/or oxygen and/or sulphur heteroatom such as, for example, pyridyl, pyrimidinyl, triazinyl, indolyl, quinolinyl, furyl, thiophenyl, oxazolyl and isoxazolyl groups.

Optionally, the catalysts may be chemically modified to incorporate coloured ligands with functionality suitable for initiation of polymerisation, for example a primary alcohol group. Thus, there may be provided a range of coloured catalysts which produce polymers, the coloration of which may be controlled by the initiator rather than the active polymerisation catalyst.

According to a second aspect of the present invention, there is provided a method for the preparation of a radiation absorbing polymer, said method comprising performing a polymerisation reaction in the presence of a catalyst according to the first aspect of the invention.

Said polymerisation reaction may be performed according to any of the standard polymerisation techniques known to the person skilled in the art, such as emulsion polymerisation, suspension polymerisation, or solution polymerisation, and may comprise either addition polymerisation or condensation polymerisation. Preferably, however, said reaction comprises a condensation polymerisation. Said reaction may be carried out in any one of batch, semi-batch or continuous mode.

Most preferably, the method according to the second aspect of the present invention comprises a condensation polymerisation, most particularly a condensation polymerisation reaction carried out for the preparation of a polyester, such as poly(ethylene terephthalate). An especially preferred embodiment of the present invention comprises the ring opening polymerisation of a lactide in the preparation of poly(lactic acid). Other preferred embodiments include the synthesis of polycaprolactone, poly(glycolic acid), and other thermoplastic polymers.

According to a third aspect of the present invention, there is provided a polymeric material prepared by means of the method according to the second aspect of the invention. Preferably, said polymeric material comprises a condensation polymer, more preferably a polyester. Most preferably, however, said polymeric material comprises poly(lactic acid). Typically, said polymeric materials have molecular weights which fall in the range of from 1,000 to 100,000, more preferably from 5,000 to 60,000.

Coloured polymeric materials according to the third aspect of the invention show good levels of colour strength and colour fastness, since the chromophoric materials are intimately involved in the process of polymer formation and are intrinsically bound to the polymer structure. Typically, the resulting polymeric materials may subsequently be melt spun into filaments, which can then be drawn into yarns for textile fibre production.

A constant concern with polymers manufactured for textile production according to the methods of the prior art has been the problem of discoloration, primarily as a consequence of the presence of unwanted catalyst in the final polymer. Such discoloration makes the reliable achievement of desired shades extremely difficult in subsequent coloration processes, and this frequently necessitates pre-coloration treatment of the polymer to ensure the removal of residual catalyst, which can be a difficult, time-consuming and expensive process. Naturally, however, such drawbacks are overcome when using the catalysts and method of the present invention, since the catalysts are intrinsically coloured and are intended to produce coloration in the polymers. Thus, the production of polymers by this method eliminates not only the requirement for post-polymer production coloration processes, but also the necessity to remove residual catalyst from the polymer.

Throughout the description and claims of this specification, the words “comprise” and “contain” and variations of the words, for example “comprising” and “comprises”, means “including but not limited to”, and is not intended to (and does not) exclude other moieties, additives, components, integers or steps.

Throughout the description and claims of this specification, the singular encompasses the plural unless the context otherwise requires. In particular, where the indefinite article is used, the specification is to be understood as contemplating plurality as well as singularity, unless the context requires otherwise.

Features, integers, characteristics, compounds, chemical moieties or groups described in conjunction with a particular aspect, embodiment or example of the invention are to be understood to be applicable to any other aspect, embodiment or example described herein unless incompatible therewith.

### DESCRIPTION OF THE INVENTION

Particularly preferred examples of catalysts according to the first aspect of the present invention comprise aluminium complexes. Especially preferred examples of such compounds comprise complexes capable of catalysing the ROP of lactide, and which allow for considerable modification of the steric and electronic properties of the ligand framework, and hence polymerisation activity.

Specifically, a range of five-coordinate aluminium complexes containing two arene-functionalised picolinamide ligands has been synthesised by standard techniques, as shown in Scheme 1. These complexes have been fully characterised and the structure for compound 1 (Scheme 1) has been confirmed by means of X-ray crystallography. This revealed a five-coordinate aluminium centre with a square pyramidal geometry. In addition, the polymerisation activity of the complexes has been determined by firstly adding benzyl alcohol activator, then evaluating the catalytic potential of the complexes with respect to rac-lactide polymerisation at 70° C. in toluene at 1.6 mol % catalyst loading. The results of this study are shown in Table 3.

TABLE 3
Lactide Polymerisation using Aluminum Picolinamide Complexes.

| | | % | | | | | |
|---|---|---|---|---|---|---|---|
| | | Conversion | | [PLA]/ | | | Poly- |
| Catalyst | X | (time/h) | | mmol | M_{w} | Mₙ | dispersity |
| 1 | p-NO₂ | 100 | (3) | 7.0 | 15,900 | 11,200 | 1.4 |
| 2 | m-NO₂ | 100 | (3) | 7.0 | 25,000 | 21,200 | 1.2 |
| 3 | 2,4,6- | 93.4 | (70) | 6.54 | 9,530 | 7,690 | 1.2 |
| | Me₃ | | | | | | |
| 4 | p-F | 100 | (46) | 7.0 | 16,300 | 11,300 | 1.4 |
| 5 | 2,4- | 96.1 | (70) | 6.73 | 6,700 | 4,300 | 1.6 |
| | (OMe)₂ | | | | | | |

During the course of the polymer synthesis reactions, the first aliquots from the polymerisation mixture were removed after three hours, revealing that for catalysts 1 and 2, essentially all of the lactide had been polymerised. It was shown that the most active catalyst, based on percentage conversion in the shortest time and the highest molecular weight/molecular number of the polymer produced, was 2, which converted 100% lactide to polylactide within three hours. The polymer produced had a molecular weight of 25,000 and molecular number of 21,200, giving a polydispersity of 1.2. These values are comparable to those achievable with the alternative catalysts of the prior art, and show great potential for further optimisation.

Catalyst 3 showed lower activity in terms of PLA molecular weight and number, and complete conversion was difficult to achieve. These observations are consistent in terms of the effect of the ligand on the electrophilicity of the aluminium centre, since the nitro group is the strongest electron withdrawing group of the substituents investigated, and provides the most active catalysts (1,2), whereas the electron donating methyl and methoxy groups in catalysts 4 and 5 result in less efficient catalysts, presumably due to increased electron density on aluminium. Thus, the potential for controlling and optimising the activity of the catalysts according to the invention is apparent.

Specifically, therefore, a particularly preferred embodiment of the present invention comprises a catalyst for use in the preparation of a coloured polymeric material, said catalyst comprising a coloured organometallic compound which comprises an aluminium complex comprising at least one picolinamide ligand. Preferably, said at least one picolinamide ligand comprises at least one arene-functionalised picolinamide ligand. Most preferably said at least one arene-functionalised picolinamide ligand comprises at least one electron withdrawing group. Particularly preferred catalysts comprise two such ligands. Said catalysts are especially useful in conjunction with PLA polymerisation reactions, and may be adapted to control all aspects of PLA polymerization.

As previously observed, a key aspect in the commercial production of polymers by the methods of the prior art is decolourisation, wherein spent catalyst is removed from the synthesised polymer. Said removal process is often difficult to carry out and, even when it is possible, the process is expensive. However, the present invention proposes the use of catalysts containing a chromophore, thereby allowing the appropriate loading of catalyst to perform the polymerization and in addition, offering the benefit of incorporating the dye required for coloration of the material.

The process is illustrated in Scheme 2 wherein a dye (e.g. 6,9 vide infra), may be incorporated in a catalyst (e.g. 7,8), used to colour a polyester material. Scheme 2 identifies two complementary processes, in the first of which the dye is retained as a ligand for the metal-terminated polymer (7), whereas with the second approach the dye is added as an initiator (typically an alcohol), and forms part of the pre-polymerisation catalyst (9), but is incorporated into the polymer through an ester linkage at the opposite end to the metal termination. Both these techniques provide polymers with directly bound dyes, but the potentially different polymerisation kinetics and profiles, offer considerable scope for optimising the overall process to give a coloured polymer having the desired properties.

Thus, certain embodiments of the present invention provides a completely novel approach to the synthesis of polymers since, instead of excluding coloured metal complexes by strategies such as avoiding conjugated ligand systems, conjugated highly coloured catalysts are deliberately employed in the synthesis procedure.

In the case of polyesters, the polymerisation processes according to the present invention are typically carried out at lower temperatures than are normally used in the dyeing process (110-130° C.), in order to avoid potential problems associated with degradation. Thus, temperatures in the range of 0-200° C., preferably 20-110° C., more preferably 20-40° C. are typically employed for polymer preparation. Favourable results have been achieved when performing the processes in the region of 70° C., at which temperature efficient high molecular weight polymer formation is observed. In this way, problems associated with polymer degradation during wet processing and catalyst removal may be conveniently eliminated.

The process of the present invention also provides significant benefits environmentally and in terms of overall efficiency, since it completely eliminates the fibre wet processing stages in the supply chain and thereby shows advantages over current practices of fibre preparation, dyeing and finishing. Water consumption is reduced, as is the energy requirement for heating water in each of the wet processing stages, which also has obvious economic benefits. Furthermore, waste dye and the requirement for subsequent effluent treatment of coloured wastewater are eliminated.

Traditionally, disperse dyes are applied to polyester fibres, and such dyeings require a so-called reduction clearing post-treatment with reducing agents such as sodium dithionite to remove surface dye. The present process again allows this treatment to be dispensed with, thereby removing the problem of effluent pollution traditionally associated with the reduction clearing process.

Preferred catalysts according to the present invention comprise organometallic aluminium complexes which comprise picolinamide ligands with appended chromophores comprising azo dyes, examples of which are illustrated in Schemes 3 and 4. In scheme 3, there are shown examples of organometallic compounds according to the first aspect of the invention wherein the azo chromophores (6-9) which impart colour to the catalyst are attached to the two picolinamide ligands and, as a consequence, the chromophores are indirectly bound to the metal atom, as in the case of the compounds of general formula (B) above, whereas in Scheme 4 the chromophoric moieties, which comprise azo (10,12), thiazole (14) and benzothiazole (16) species are all directly bound to the metal atom as in the compounds of formula (A) above.

In each case, the catalysts are prepared from the corresponding amide or azo compound and AlMe₃, which has been found to be a particularly clean and high yielding reaction for formation of the aluminium alkyl species, although alternative procedures, such as treatment of the amide or azo compound with, for example, KH then MeAlCl₂ have also been investigated and found to be satisfactory.

Once the aluminium alkyl species has been synthesised, polymer formation is achieved by adding an alcohol initiator, typically benzyl alcohol, followed by addition of the polyester precursor, or precursors; preferably, said precursor comprises a lactide.

The three main strategies for incorporation of the dye into the catalyst structure, as previously noted comprise the following:
(1) Appending a chromophore to a ligand (compounds of formula (A));
(2) Using a chromophore as the ligand (compounds of formula (B)); or
(3) Using a chromophore as the initiator.

The invention will now be further illustrated by specific reference to each of these three alternative approaches.

### 1 Appending a Chromophore to a Ligand

Modification of the ligand framework may be achieved through amide bond formation between an appropriate nitrogen heterocycle, and an azo-dye containing a free amine, as shown in Scheme 3. The dye structures illustrated are typical azo dyes, having the colours indicated, although a very wide range of other potential dyes are available and can be accessed through the Colour Index International database. The compounds illustrated should by no means be taken as limiting the scope of the invention in any way, since it will be apparent to the skilled person that a range of acid chlorides may be combined with various amine dyes in the manner indicated in Scheme 3.²⁸

It is well known that dyes based on the azobenzene chromophore can be switched between two geometric isomers using light of suitable wavelength.²⁹ Such photoisomerisation reactions are usually rapid, reversible and of high quantum yield. It has been found that, upon isomerisation, changes in optical, mechanical and chemical properties of the azo dye unit can impart similar changes to metal complexes, polymers and surfaces.³⁰ Indeed, photoisomerisation can lead to new catalyst structures, as shown in Scheme 5, wherein compounds 17-ct and 17-cc may be obtained by carrying out the polymerisation in the presence of a suitable UV/visible radiation source with azo units in the photostationary state; these compounds 17-ct and 17-cc have different chemical and physical properties to the 17-tt ground state of the catalyst, thereby further enhancing the versatility of the present invention. The polymers obtained from polymerisation reactions involving the catalysts can undergo similar switching, allowing access to functional polymers which also have the benefit of being renewable and biodegradable, with applications in non-linear optics, and optoelectronics, and optical information storage.^{31, 32}

### 2 Using a Chromophore as the Ligand

An alternative approach is to directly use chromophores closely related to dyes (6-9) as ligands if they have the appropriate functionality. By means of this method, complexation of a chromophore to a metal such as Al may cause a broadening of the absorption spectrum of the colorants, since the conjugated system is altered through complexation with the metal, but this could be particularly advantageous to the coloration process. Dark and dull colours are usually achieved by complexation to a coordinating metal, but these complexes are too large to be applied to polyesters and PLA by standard means, due to the molecular size preventing diffusion into the relatively small areas of free volume between the polymer chains. By using the method of the present invention, however, such problems are eliminated, since the metal complex colorant comprises an integral part of the polymer, by virtue of the method of preparation.

Catalysts of this type are illustrated in Scheme 4 and, again, many suitable materials are based on classical azo-dyes (6-9), which can be part of the metal ligand binding motif (11,13). The azo dye units can be prepared using the standard procedures of the prior art, with minor modification when necessary.³³⁻³⁸ Alternatively, the azo group may be replaced with an amido function to relay conjugation (e.g. 14,18, cf. 7,9), which also allows for effective metal complexation.

### 3 Using a Chromophore as the Initiator

By simple chemical modification of existing dye structures, in order to incorporate functionality required for initiation, e.g. a primary alcohol group, it is possible to obtain chromophoric polymerisation initiators. Thus, existing catalysts, or future improved systems which do not contain relevant chromophores, may be combined with coloured initiators to give a range of active catalysts. In such systems, the chromophoric unit becomes more remote from the reaction centre as the polymerisation ensues, which is a feature that may be particularly useful. In an extension of this concept, the use of coloured catalysts in combination with coloured initiators provides further opportunity for enhancing the colour and intensity of polymers.

By use of any of these three approaches for incorporation of the dye into the catalyst structure, it is found that very satisfactory dyed polymers, showing high colour strength and fastness, may be obtained. Unoptimised molecular weights of up to 25,000 g mol⁻¹ may be achieved for PLA polymers using picolinamide catalysts at 1.6 mol % loading. From a consideration of the structures of the resultant polymers, each catalyst molecule can have an associated polymer of 25,000 g mol⁻¹ associated with it, and each dye chromophore moiety (e.g. 6-9) has a molecular weight in the region of 250-350 g mol⁻¹. The concentration ranges of dyes currently used for PLA using standard prior art procedures are 0.2-3.0% on mass of polymer, and the values achieved by means of the present invention are well within this range. Thus, a catalyst which incorporated one dye chromophore moiety would yield colorant by mass of 1.0-1.6% with respect to mass of polymer, whilst a catalyst which incorporated two dye chromophore moieties would provide 2.0-3.2% dye on mass of polymer.

An additional benefit of the present invention is that by incorporating the dye molecule at the polymer synthesis stage the colorant will be homogenous throughout the cross-section of any fibre produced. This will result in higher colour strength when compared with dyeings achieved by means of aqueous exhaustion procedures, where adsorption and diffusion mechanisms, essentially through a cylinder of polymer (fibre), do not necessarily yield complete dye homogeneity through the fibre cross-section.

The coloured PLA resins resulting from the process of the present invention may be melt-spun into filaments and the as-spun filament yarns can then be drawn using standard procedures and apparatus. The fibres which are produced show improved fastness properties when compared with their aqueous dyed counterparts. Specifically, wash fastness is increased as a consequence of the colorant being covalently bound to the polymer, whereas with aqueous dyeings the colorant occupies free volume between polymer chains, interacting via weaker van der Waals, induced dipole and hydrogen bonding forces. In addition, light fastness increases in view of the fact that the susceptible chromophore is protected within the catalyst structure.

In a particularly preferred embodiment, the present invention is applicable to the preparation of poly(lactic acid), which is a particularly environmentally friendly polymer in terms of sustainability and degradation issues. Furthermore, the process of present invention provides significant advantages over the methods of the prior art in the light of the reduced reaction temperature and the elimination of the need for decolorisation and subsequent dyeing procedures, thereby greatly improving the sustainability of the overall technology in terms of cost and environmental impact.

Poly(lactic acid) is expected to become increasingly important as a sustainable textile polymer through the 21^{st} century, and its increasing use will ease the pressure on fossil fuel resources and actively decrease atmospheric carbon dioxide levels³⁹. A successful PLA coloration system, as provided by the current invention, will overcome the current shortcomings of aqueous dyed PLA, reduce the cost of PLA processing, and fulfil all the technical requirements for apparel and related uses to afford an economic, sustainable, feasible replacement for standard polyesters.

Various aspects of the present invention will now be further illustrated, though without in any way limiting the scope of the invention, by reference to the following examples.

### EXAMPLES

### Syntheses of Catalysts

All syntheses of catalysts are carried out under an atmosphere of dry dinitrogen using dry solvents.

The general scheme for the preparation of aluminium-based catalysts is as follows:

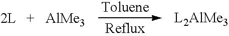

wherein L=dye ligand.

### Example 1

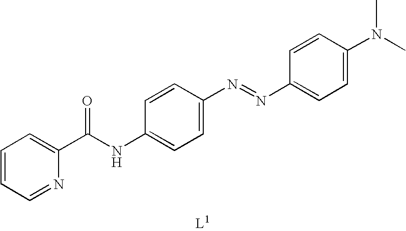

Trimethylaluminium (0.08 cm³, 0.8 mmol) was added to a suspension of L¹ (0.52 g, 1.5 mmol) in toluene (40 cm³). The reaction was heated under reflux overnight, and then cooled to room temperature to yield a dark orange solution and precipitate. The mixture was filtered, the solvent removed in vacuo and the residue washed with petrol to yield a red solid, catalyst C1.

### Example 2

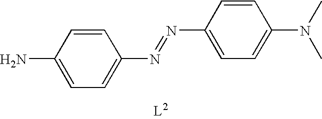

Trimethylaluminium (0.20 cm³, 2.1 mmol) was added to a suspension of L² (4′-amino-N,N-dimethyl-4-aminoazobenzene; C. I. Disperse Black 3; 1.00 g, 4.2 mmol) in toluene (40 cm³). The reaction was heated under reflux overnight, and then cooled to room temperature to yield a dark red solution and precipitate. The mixture was filtered, the solvent removed in vacuo and the residue washed with petrol to yield a black solid, catalyst C2.

### Example 3

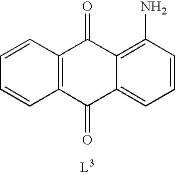

Trimethylaluminium (0.22 cm³, 2.3 mmol) was added to a suspension of L³ (1-aminoanthraquinone; 1.00 g, 4.5 mmol) in toluene (40 cm³). The reaction was heated under reflux overnight, and then cooled to room temperature to yield a pale solution and dark purple precipitate. The solid was isolated by filtration, washed with THF and acetonitrile and dried in vacuo to yield a black solid, catalyst C3.

### Example 4

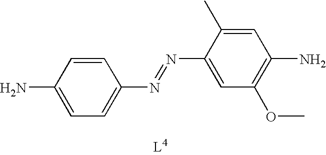

Trimethylaluminium (0.19 cm³, 2.3 mmol) was added to a suspension of L⁴ (4,4′-diamino-2-methyl-5-methoxyazobenzene; C. I. Disperse Black 2; 1.00 g, 3.9 mmol) in toluene (40 cm³). The reaction was heated under reflux overnight, and then cooled to room temperature to yield a pale solution and black precipitate. The solid was isolated by filtration, washed with petrol and dried in vacuo to yield a black solid, catalyst C4.

### Example 5

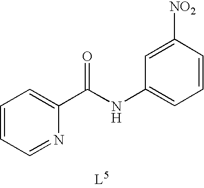

Trimethylaluminium (0.15 cm³, 1.6 mmol) was added to a suspension of L⁵ (N-(3-nitrophenyl)-2-pyridinecarboxamide; 0.70 g, 2.9 mmol) in toluene (40 cm³). The reaction was heated under reflux overnight, and then cooled to room temperature to yield a dark orange solution and brown precipitate. The mixture was filtered, the solvent removed in vacuo and the residue washed with petrol to yield an orange solid, catalyst C5.

### Syntheses of Polymers

### Polyesters

The general scheme for the preparation of poly(ethylene terephthalate) is as follows:

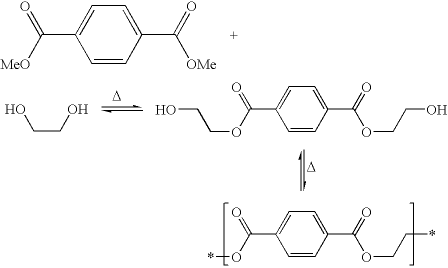

### Example 6

A mixture of catalyst C5 (0.05 g, 0.1 mmol), dimethyl terephthalate (2 g, 10.3 mmol) and ethylene glycol (1.5 g, 24.2 mmol) was heated at 210° C. for 4 hours, then under reduced pressure at 280° C. for a further 2 hours to yield polyethylene terephthalate (PET).

### Polymerisation of Cis-Lactide

All polymerisation reactions are carried out under an atmosphere of dry dinitrogen using dry solvents.

The general scheme for the polymerisation of cis-lactide is as follows:

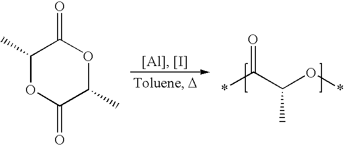

wherein [Al]=aluminium catalyst, [I]=polymerisation initiator.

Poly(lactic acid) (PLA) was characterised by ¹H NMR spectroscopy, which shows a good separation between monomer and polymer signals.¹³

### Example 7

A mixture of catalyst C1 (0.08 g, 0.1 mmol), cis-lactide (1.00 g, 6.9 mmol) and benzyl alcohol (0.02 cm³, 0.2 mmol) in toluene (30 cm³) was heated to 80° C. for 68 hours. The reaction was quenched by rapid cooling in liquid nitrogen, the solvent removed in vacuo, and the residue dissolved in dichloromethane. PLA precipitated on addition of methanol followed by storage at −18° C., and was isolated by filtration, washed with methanol and water and dried to yield an orange polymer.

Analytical Data: ¹H NMR (CDCl₃), δ (ppm): 5.20, multiplet. M_{w}: 2000-5000 by ES-MS. λₘₐₓ (nm), ε (m² g⁻¹) in DCM: 427, 0.150; 319, 0.079; 258, 0.096

### Example 8

A mixture of catalyst C2 (0.13 g), cis-lactide (1.00 g, 6.9 mmol) and benzyl alcohol (0.02 cm³, 0.2 mmol) in toluene (30 cm³) was heated to 80° C. for 19 hours. The reaction was quenched by rapid cooling in liquid nitrogen, the solvent removed in vacuo, and the residue dissolved in dichloromethane. PLA precipitated on addition of methanol followed by storage at −18° C., and was isolated by filtration, washed with methanol and water and dried to yield an orange polymer.

Analytical Data: ¹H NMR (CDCl₃), δ (ppm): 5.20, multiplet. M_{w}: 1200-2200 by ES-MS and MALDI-TOF Melting point: 111.2° C. (DSC) □H_{f}: 37.99 J g⁻¹. % Crystallinity: 40.8. λₘₐₓ (nm), ε (m² g⁻¹) in DCM: 422, 0.322; 302 (shoulder), 0.118; 259, 0.159.

### Example 9

A mixture of catalyst C3 (0.15 g), cis-lactide (1.00 g, 6.9 mmol) and benzyl alcohol (0.015 cm³, 0.15 mmol) in toluene (30 cm³) was heated to 80° C. for 162 hours. The reaction was quenched by rapid cooling in liquid nitrogen, the solvent removed in vacuo, and the residue dissolved in dichloromethane. PLA precipitated on addition of methanol followed by storage at −18° C., and was isolated by filtration, washed with methanol and water and dried to yield a brown polymer.

Analytical Data: ¹H NMR (CDCl₃), δ (ppm): 5.20, multiplet. M_{w}: 1800-3200 by ES-MS and MALDI-TOF. Melting point: 138.0° C. (DSC). ΔH_{f}: 30.05 J g⁻¹. % Crystallinity: 32.3. λₘₐₓ (nm), ε (m² g⁻¹) in DCM: 404, 0.071; 280 (shoulder), 0.210; 246, 0.317.

### Example 10

A mixture of catalyst C4 (0.1 g), cis-lactide (1.00 g, 6.9 mmol) and benzyl alcohol (0.02 cm³, 0.2 mmol) in toluene (30 cm³) was heated to 80° C. for 285 hours. The reaction was quenched by rapid cooling in liquid nitrogen, the solvent removed in vacuo, and the residue dissolved in dichloromethane. PLA precipitated on addition of methanol followed by storage at −18° C., and was isolated by filtration, washed with methanol and water and dried to yield an orange/brown polymer.

Analytical Data: ¹H NMR (CDCl₃), δ (ppm): 5.20, multiplet. M_{w}: 1500-5500 by ES-MS and MALDI-TOF. Melting point: 132.8° C. (DSC). ΔH_{f}: 25.81 J g⁻¹. % Crystallinity: 27.8. λₘₐₓ (nm), ε (m² g⁻¹) in DCM: 392, 0.309; 302, 0.249.

### Example 11

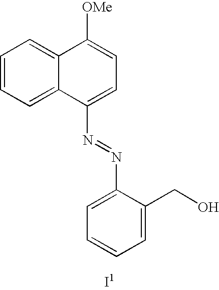

A mixture of catalyst C5 (0.03 g, 0.06 mmol), cis-lactide (0.50 g, 3.5 mmol) and I¹ (0.017 g, 0.06 mmol) in toluene (30 cm³) was heated to 80° C. for 20 hours. The reaction was quenched by rapid cooling in liquid nitrogen, the solvent removed in vacuo, and the residue dissolved in dichloromethane. PLA precipitated on addition of methanol followed by storage at −18° C., and was isolated by filtration, washed with methanol and water and dried to yield an orange/brown polymer.

### REFERENCES

¹ R. E. Drumright, P. R. Gruber, and D. E. Henton, Advanced Materials (Weinheim, Germany), 2000, 12, 1841.
² H. Tsuji and Y. Ikada, Journal of Applied Polymer Science, 1998, 67, 405.
³ E. S. Lipinsky and R. G. Sinclair, Chemical Engineering Progress, 1986, 82, 26.
⁴ M. Vert, G. Schwarch, and J. Coudane, Journal of Macromolecular Science, Pure and Applied Chemistry, 1995, A32, 787.
⁵ N. L. http://www.ingeofibers.com/ingeo/home.asp.
⁶ J. Lunt, Polymer Degradation and Stability, 1998, 59, 145.
⁷ Z. Tang, X. Chen, X. Pang, Y. Yang, X. Zhang, and X. Jing, Biomacromolecules, 2004, 5, 965.
⁸ J. Lunt and J. Bone, AATCC Review, 2001, 1, 20.
⁹ O. Dechy-Cabaret, B. Martin-Vaca, and D. Bourissou, Chem. Rev., 2004, 104, 6147.
¹⁰ D. Mecerreyes and R. Jerome, Macromolecular Chemistry and Physics, 1999, 200, 2581
¹¹ W. M. Stevels, P. J. Dijkstra, and J. Feijen, Trends Polym. Sci., 1997, 5, 300
¹² B. J. O'Keefe, S. M. Monnier, M. A. Hillmyer, and W. B. Tolman, J. Am. Chem. Soc., 2001, 123, 339
¹³ N. Spassky, M. Wisniewski, C. Pluta and A. L. Borgne, Macromolecular Chemistry and Physics, 1996, 197, 2627
¹⁴ C. P. Radano, G. L. Baker, and M. R. Smith, J. Am. Chem. Soc., 2000, 122, 1552
¹⁵ T. M. Ovitt and G. W. Coates, J. Am. Chem. Soc., 2002, 124, 1316
¹⁶ T. M. Ovitt and G. W. Coates, Journal of Polymer Science, Part A: Polymer Chemistry, 2000, 38, 4686
¹⁷ P. Hormnirun, E. L. Marshall, V. C. Gibson, A. J. P. White, and D. J. Williams, J. Am. Chem. Soc., 2004, 126, 2688
¹⁸ P. A. Cameron, D. Jhurry, V. C. Gibson, A. J. P. White, and D. J. Williams, Macromol. Rapid Commun., 1999, 20, 616
¹⁹ S. Doherty, R. J. Errington, N. Housley, and W. Clegg, Organometallics, 2004, 5, 965.
²⁰ L. M. Alcazar-Roman, B. J. O'Keefe, M. A. Hillmyer, and W. B. Tolman, Dalton Transactions, 2003, 3082
²¹ J. Lewinski, J. Zachara, B. Mank, and S. Pasynkiewicz, J. Organomet. Chem., 1993, 454, 5
²² L. K. Burdsall, A. L. Gott, and P. C. McGowan, Unpublished Work.
²³ R. S. Blackburn, X. Zhao, and D. Farrington, Dyes and Pigments, 2006, 71, 18.
²⁴ R. S. Blackburn, D. Farrington, and X. Zhao, Polymer Preprints (American Chemical Society, Division of Polymer Chemistry), 2004, 45, 600.
²⁵ Y. Yang and S. Huda, AATCC Review, 2003, 3, 56.
²⁶ T. Bechtold, A. Turcanu, A. Gangiberger, and E. Geissler, Journal of Cleaner Production, 2003, 11, 499.
²⁷ H. T. Deo and B. K. Desai, Journal of the Society of Dyers and Colourists, 1999, 115, 224.
²⁸ S. B. Mhaske and N. P. Argade, Journal of Organic Chemistry, 2004, 69, 4563.
²⁹ H. Knoll, ‘Photoisomerisation of Azobenzenes’, in Organic Photochemistry and Photobiology, ed. W. M. Horspool and F. Lenci, CRC Press, Boca Raton, 2003, 89.1.
³⁰ R. A. vanDelden, T. Mecca, C. Rosini, and B. L. Feringa, Chem. Eur. J., 2004, 10, 61.
³¹ A. Altomare, F. Ciardelli, L. Mellini, and R. Solaro, Macromolecular Chemistry and Physics, 2004, 205, 1611.
³² R. H. El Halabieh, O. Mermut, and C. J. Barrett, Pure and Applied Chemistry, 2004, 76, 1445.
³³ M. Matsui, Y. Kamino, M. Hayashi, K. Funabiki, K. Shibata, H. Muramatsu, Y. Abe, and M. Kaneko, Liquid Crystals, 1998, 25, 235.
³⁴ V. Rajendran and M. J. Nanjan, Journal of Polymer Science, Part A: Polymer Chemistry, 1987, 25, 829.
³⁵ J.-H. Choi, S.-H. Hong, and A. D. Towns, J. Soc. Dyers and Colourists, 1999, 115, 32.
³⁶ D. Cledat, J. C. Bollinger, H. Hoja, J. Debord, and B. Penicaut, Quantitative Structure-Activity Relationships, 1998, 17, 1.
³⁷ Y. Gok and B. Senturk, Organic Preparations and Procedures International, 1995, 27, 87.
³⁸ V. P. Dedkova, M. A. Azarashvili, and S. B. Sawin, Zhurnal Analiticheskoi Khimii, 1989, 44, 1246.
³⁹ R. S. Blackburn, D. W. Farrington, J. Lunt, and S. Davies, Poly(lactic acid) in Biodegradable and Sustainable Fibres, R. S. Blackburn (Ed.), Chapter 6, Woodhead Publishing, 2005.

1. A catalyst for use in the preparation of a radiation absorbing polymeric material, said catalyst comprising a radiation absorbing organometallic compound, wherein the wavelength of maximum absorption of each of said radiation absorbing polymeric material and said radiation absorbing organometallic compound lies in the region of from 200-1200 nm.

2. The catalyst as claimed in claim 1 which comprises a polymerisation catalyst.

3. The catalyst as claimed in claim 2 wherein said radiation absorbing polymeric materials and said radiation absorbing organometallic compounds have a wavelength of maximum absorption in the infra-red region, ultra-violet region or visible region of the electromagnetic spectrum.

4-5. (canceled)

6. The catalyst as claimed in claim 1 which comprises at least one organic chromophore and at least one metal atom.

7. The catalyst as claimed in claim 6 wherein said metal comprises a transition metal, a lanthanide or an actinide.

8. (canceled)

9. The catalyst as claimed in claim 6 wherein said metal comprises aluminium.

10. (canceled)

11. The catalyst as claimed in claim 6 wherein said chromophore is selected from azo compounds, di- and tri-arylmethane compounds, methine, polymethine and azomethine derivatives, anthraquinone compounds, phthalocyanine derivatives, xanthene, acridine, azine, oxazine, thiazine, indamine, indophenol, aminoketone, hydroxyketone, nitro, nitroso, quinoline, stilbene and thiazole compounds, and carbocyclic and heterocyclic derivatives.

12. (canceled)

13. The catalyst as claimed in claim 1 wherein said organometallic compound comprises a metal complex compound wherein the metal atom is attached to at least one ligand.

14. The catalyst as claimed in claim 13 wherein said metal atom is attached to two ligands.

15. The catalyst as claimed in claim 13 which comprises a coloured compound of the general formula (A):
MLₓD_{y} (A)
wherein D represents a chromophoric group;
M represents a metal atom; L represents a non-chromophoric ligand; x=0-8; and y=1-9.

16. The catalyst as claimed in claim 6 which comprises a compound of formula (B)
D-M-D (B)
wherein D and M have the meanings previously ascribed to them,
the chromophoric groups may be the same or different, and said chromophoric groups comprise a ligand which is attached to said metal atom, said chromophoric groups thereby being directly bound to said metal atom.

17. The catalyst as claimed in claim 6 which comprises a compound of formula (B-1):
D¹-M-D² (B-1)
wherein D¹ and D² represent chromophoric groups D which may be the same or different; and
M represents a metal atom.

18. The catalyst as claimed in claim 13 which comprises a compound of formula (C)
D-M-L (C)
wherein D and M have the meanings previously ascribed to them, and said chromophoric group comprises a ligand which is attached to said metal atom, said chromophoric group thereby being directly bound to said metal atom, wherein said ligand comprises an organic residue and wherein said organic residue comprises an aryl or heteroaryl residue.

19. The catalyst as claimed in claim 14 which comprises a compound of formula (D)
D-L-M-L-D (D)
wherein D and M have the meanings previously ascribed to them, the chromophoric groups and non-chromophoric ligands may be the same or different, and said chromophoric groups are attached to said non-chromophoric ligands said chromophoric groups thereby being indirectly bound to said metal atom, wherein said ligand comprises an organic residue, and wherein said organic residue comprises an aryl or heteroaryl residue.

20. The catalyst as claimed in claim 19 which comprises a compound of formula (D-1):
D¹-L¹-M-L²-D² (D-1)
wherein D¹ and D² represent chromophoric groups D which may be the same or different;
M represents a metal atom; and
L¹ and L² represent non-chromophoric ligands L which may be the same or different, wherein said ligand comprises an organic residue and wherein said organic residue comprises an aryl or heteroaryl residue.

21. The catalyst as claimed in claim 14 which comprises a compound of formula (E)
D-L-M-D (E)
wherein D and M have the meanings previously ascribed to them, the chromophoric groups may be the same or different, and a first of said chromophoric groups is attached to said non-chromophoric ligand, said first chromophoric group thereby being indirectly bound to said metal atom, whilst a second of said chromophoric groups comprises a ligand which is attached to said metal atom, said second chromophoric group thereby being directly bound to said metal atom, wherein said ligand comprises an organic residue.

22. The catalyst as claimed in claim 21 which comprises a compound of formula (E-1):
D¹-L-M-D² (E-1)
wherein D¹ and D² represent chromophoric groups D which may be the same or different, D¹ representing said first chromophoric group which is attached to said non-chromophoric ligand, said first chromophoric group thereby being indirectly bound to said metal atom, whilst D² represents said second chromophoric group, which comprises a ligand which is attached to said metal atom, said second chromophoric group thereby being directly bound to said metal atom, M represents a metal atom; and L represents a non-chromophoric ligand, wherein said ligand comprises an organic residue, and wherein said organic residue comprises an aryl or heteroaryl residue.

23-24. (canceled)

25. The catalyst as claimed in claim 19 wherein said aryl residue comprises a phenyl, naphthyl, anthracyl or phenanthryl residue.

26. The catalyst as claimed in claim 25 wherein said heteroaryl residue comprises a heterocycle containing at least one nitrogen and/or oxygen and/or sulphur heteroatom.

27. The catalyst as claimed in claim 26 wherein said heteroaryl residue comprises a pyridyl, pyrimidinyl, triazinyl, indolyl, quinolinyl, furyl, thiophenyl, oxazoiyl or isoxazolyl residue.

28-29. (canceled)

30. The catalyst as claimed in claim 26 wherein said nitrogen or oxygen-containing group comprises an amino group or a hydroxy group.

31. The catalyst as claimed in claim 1 which comprises at least one picolinamide ligand.

32. The catalyst as claimed in claim 31 which comprises at least one arene-functionalised picolinamide ligand.

33. The catalyst as claimed in claim 32 wherein said arene-functionalised picolinamide ligand comprises at least one electron withdrawing group.

34. The catalyst as claimed in claim 31 which comprises two picolinamide ligands.

35. The catalyst as claimed in claim 34 which is chemically modified to incorporate functionality suitable for initiation of polymerisation.

36. The catalyst as claimed in claim 35 wherein said chemical modification comprises the incorporation of a primary alcohol group.

37. A method for the preparation of a radiation absorbing polymer, said method comprising performing a polymerisation reaction in the presence of a catalyst as claimed in claim 1.

38. The method as claimed in claim 37 wherein said radiation absorbing polymer comprises a polymer having a wavelength of maximum absorption in the infra-red region, ultraviolet region or visible region of the electromagnetic spectrum.

39-44. (canceled)

45. The method as claimed in claim 37 which comprises a ring opening polymerisation of a lactide.

46. The method as claimed in claim 45 which comprises the preparation of poly(lactic acid).

47. The method as claimed in claim 37 which comprises the preparation of polycaprolactone or poly(glycolic acid).

48. A radiation absorbing polymeric material prepared according to the method as claimed in claim 37.

49-60. (canceled)
